(19) United States
(12) Patent Application Publication
(10) Pub. No.: US 2009/0038555 A1
(43) Pub. Date: Feb. 12, 2009

(54) Animal scent detection apparatus and system

(76) Inventor: David Reese, Forest Park, IL (US)

Correspondence Address: Timothy J. Haller;Niro, Scavone, Haller & Niro, Suite 4600, 181 West Madison Street, Chicago, IL, 60602, US

(21) Appl. No.: 11/891,176

(22) Filed: Aug. 9, 2007

(51) Int. Cl. A01K 29/00; G01N 33/00

(52) U.S. Cl. 119/174; 73/23.34

(57) ABSTRACT

A portable scent detection system for moving air from a search area to an animal's nose that can include a hand-held pickup for collecting air from a target area, an air moving unit containing at least one fan, a return hose coupled between the pickup and the air moving unit, and a scent presentation device worn on the animal's head with a one or more presentation ports near the animal's nose. The preferred animal is a dog; however, any animal which can be trained to indicate detection of a target scent can be used. The air moving unit can be double or single. A single unit can contain one fan, while a double unit can contain a second fan. More fans can be added in series or parallel as needed to provide adequate airflow. The pickup device can also optionally contain a fan. The fans can be battery powered using fixed or rechargeable batteries. The battery or batteries can be external or internal to the unit. The air moving unit can be contained in a waist or backpack for ease in use. The scent pickup can also optionally contain a filter to filter out dust or unwanted particles. A system of hoses normally can be run between the pickup device and the air moving unit and between the air moving unit and the scent presentation device. The air moving unit can optionally be located in a widened section of the hoses or hoses.

### BACKGROUND

1. Field of the Invention

The present invention relates generally to the field of scent detection and more particularly to a animal scent detection system.

2. Description of the Prior Art

It is well known to use dogs or other animals to search for contraband such as drugs or explosives. This is because animals have noses that are far more sensitive and responsive to a wide variety of materials than humans. Dogs, for example, are know to be able to detect various materials in the parts per billion range. Canines have been used particularly because they can be trained to respond in a predetermined manner when they detect the presence of a target substance.

In the modern world, it is becoming more and more important to be able to detect drugs, explosives and other contraband that produces a detectable scent. This is particularly true at airport security check points, national border entry points and other key locations where people and vehicles need to be checked. It is also important to law enforcement personnel to be able to find hidden contraband in premises or business searches or find explosives of contraband in containers and vehicles.

One problem with simply having a dog sniff for substances in hand carried luggage or at a check point is that such a procedure may annoy or frighten an innocent person, or it might even be an unacceptable invasion of privacy. This is particularly true in a place like an airport security check point where numerous people of all ages and genders routinely pass through. A second problem arises in buildings where target substances may be hidden out of smelling range of an animal walking on a leash (contraband hidden above ceiling tiles for example). This problem might even arise with persons passing through a check point if the substance was hidden on their body high enough to put it above the dog's smelling range.

It is known in the art to use a pass-through portal with air taken from the portal and passed to a dog or a machine. However, this technique is not portable for use in building searches, and it generally requires the dog to stay very near one point in the equipment such as an opening where air samples are directed.

It would be advantageous to have a portable apparatus that would allow security and law enforcement personnel to take advantage of the sensitivity and selectivity of a canine's nose (or any other animal) to detect trace scent from target materials such as explosives, drugs and the like while walking with the dog through the search area.

### SUMMARY OF THE INVENTION

The present invention relates to a portable scent detection system for moving air from a search area to an animal's nose that can include a hand-held pickup for collecting air from a target area, an air moving unit containing at least one fan, a return hose coupled between the pickup and the air moving unit, and a scent presentation device worn on the animal's head with a one or more presentation ports near the animal's nose. The preferred animal is a dog or canine; however, any animal which can be trained to indicate detection of a target scent can be used. The air moving unit can be double or single. A single unit can contain one fan, while a double unit can contain a second fan. More fans can be added in series or parallel as needed to provide adequate airflow. The pickup device can also optionally contain a fan. The fans can be battery powered using fixed or rechargeable batteries. The battery or batteries can be external or internal to the unit. The air moving unit can be contained in a waist or backpack for ease in use. The scent pickup can also optionally contain a filter to filter out dust or unwanted particles. A system of hoses normally can be run between the pickup device and the air moving unit and between the air moving unit and the scent presentation device. The air moving unit can optionally be located in a widened section of the hoses or hoses.

The system is normally used by attaching the air moving unit and batteries in a waist or backpack with the scent presentation device strapped to a canine's nose. The operator and animal can then sweep and area using the pickup device to direct scents to the canine. The animal can be trained to appropriately signal when a target scent is detected.

### DESCRIPTION OF THE DRAWINGS

Several drawings are being provided to illustrate embodiments of the present invention.

FIG. 1 shows a dog being used with a portable embodiment of the present invention to detect scents.

FIG. 2 shows a muzzle-like device that can be worn by a dog or other animal that can direct scents to the animal's nose.

FIG. 3 shows details of a portable embodiment of the present invention.

FIG. 4 shows internal details, including the possible location of fans, of the embodiment of FIG. 3

FIG. 5 shows a different embodiment of a portable unit.

FIG. 6 shows an embodiment of the invention with a fan embedded in an enlarged portion of tubing.

FIG. 7 shows a optional auxiliary fan unit that can be attached to embodiments of the invention for searching larger areas such as containers, truck cargo bays and the like.

Several drawings and illustrations have been presented to aid in understanding the present invention. The scope of the present invention is not limited to the embodiments or details shown in the figures.

### DESCRIPTION OF THE INVENTION

The present invention relates generally to an apparatus and system for directing scents to a dog's or other animal's nose so that the natural sensitivity and selectivity of the animal's nose can be used to detect concealed target substances such as drugs or explosives.

One of the objects of the present invention is to pick up air from the immediate vicinity of the target substance and move that air to the animal's nose in such a way that the search area can be at any height, and done in a way that the animal can move around freely. The term dog or canine will be used in this application to represent any animal that can be trained and used to respond to scent. While dogs are the most common animals that can be used in this application, and are preferred because of their intelligence and ability to be trained, any other animal that can be trained to respond may be used and will be included under the name canine or dog in the present application.

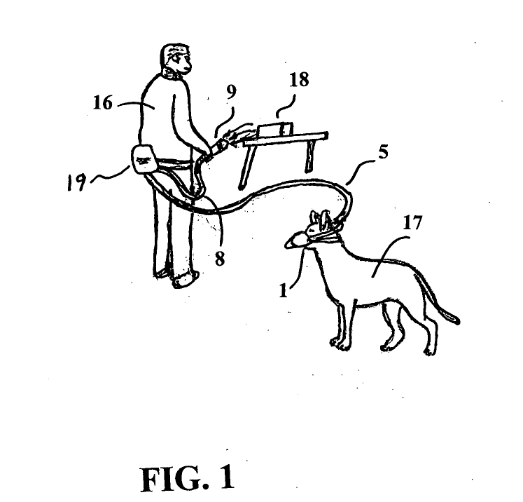

A particular embodiment of the present invention includes a muzzle-like device that fits over the dog's snout and can be loosely and comfortably strapped to its head. Air samples taken from a search area can be directed into a fan unit and through tubing to this device. FIG. 1 shows an example of this embodiment. A dog 17 wearing such a device 1 cooperates with a human operator 16 who wears a portable fan and battery unit 19 in a waist pack. Searching is performed by the operator 16 using a hose 8 and inlet pickup device 9. The small portable unit 19 runs on batteries and moves air from the pickup device 9 to the scent presentation device 1 on the dog's nose via a tube or hose 5. The dog can be trained to respond in a predetermined manner such as sitting upon detection of a target scent. The device can be operated by placing it on the dog, and starting the fans making generally sure that fresh air (with no contamination from any target substance) is entering the pickup. After airflow is established, the operator and dog can proceed to a search area. The portable unit 19 can be worn by the operator during the search. The operator-dog team can then proceed on foot to perform the search for contraband 18. While FIG. 1 shows a item on a table to aid in the understanding of the invention, in reality, contraband 18 may be hidden in walls or ceilings, under beds, in cabinets or anywhere.

A room can be searched quickly using the apparatus of the present invention by moving the pickup to the vicinity of points of interest including the walls or ceiling. The dog can be trained to give an indication of a detection by either moving its body (sitting for example), or by making some other type of indication. Training of this type is unique in itself. Generally when a detection is made, the operator can optionally move away from the locus with the dog, make sure the pickup is pulling in only clean air at that point, wait until the dog has stopped responding, and then return to the suspect area. If the dog gives a second indication of a hit or detection, the operator or other personnel can search for the actual contraband.

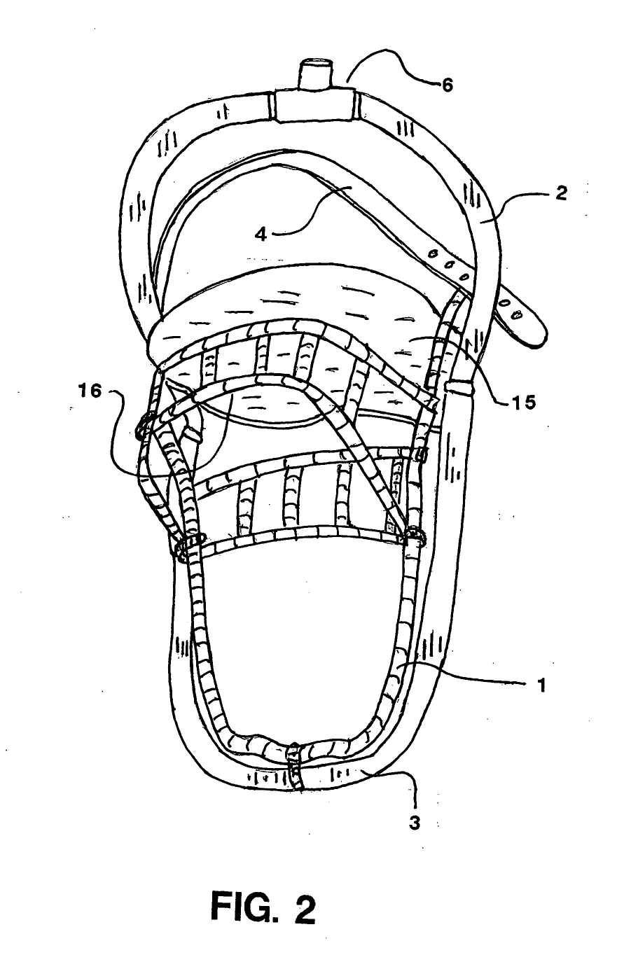

FIG. 2 shows a detail of an embodiment of the muzzle-like scent presentation device that can be worn by the dog. The device can have a rigid metal or plastic frame 1 that is shaped to fit over an animals snout so that the nose is near the front of the device. The device can include an over-bar 16 that passes over the nose. Incoming airflow can enter a T-junction 6 that directs the flow into tubing 2 on each side. While tubing is shown in FIG. 2 on both sides, tubing on only one side is within the scope of the present invention. Holes 3 in the tubing 2 direct the flow to the nose. The holes or other presentation ports or means 3 can be far enough away from the nose so that the dog is comfortable and yet can detect target substances in the desired sensitivity range. The device can be fitted onto the animal's head with a harness 15 that can attach with one or more straps 4. The harness 15 and strap(s) 4 can be made from leather, artificial leather or any other flexible material. The strap can contain holes that mate into a buckle.

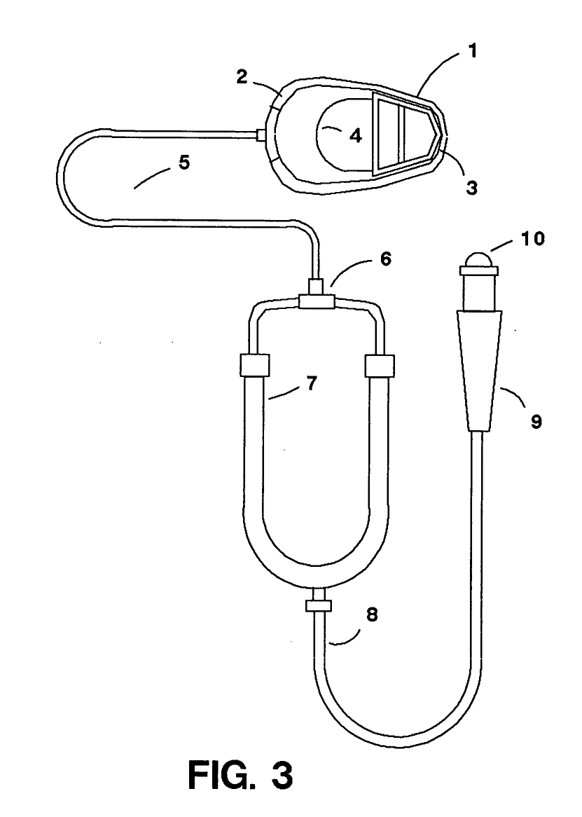

FIG. 3 shows an embodiment of a portable apparatus using the principles of the present invention. A central portable unit 7 can contain fans and provide an air moving point to pull air from an inlet hose 8 and pickup 9. Air can move through the portable unit 7 and be forced out both sides to a junction 6 where it can enter a single feed hose 5. The feed hose 5 can be coupled to the muzzle-like device 1 where air is taken through the one or more feed hoses 2 to a section with exit holes or other presentation ports 3. The muzzle-like device 1 can be held on the animal's head with a harness and one or more straps 4. Air entering the entrance 10 of the pickup 9 can optionally be filtered if desired.

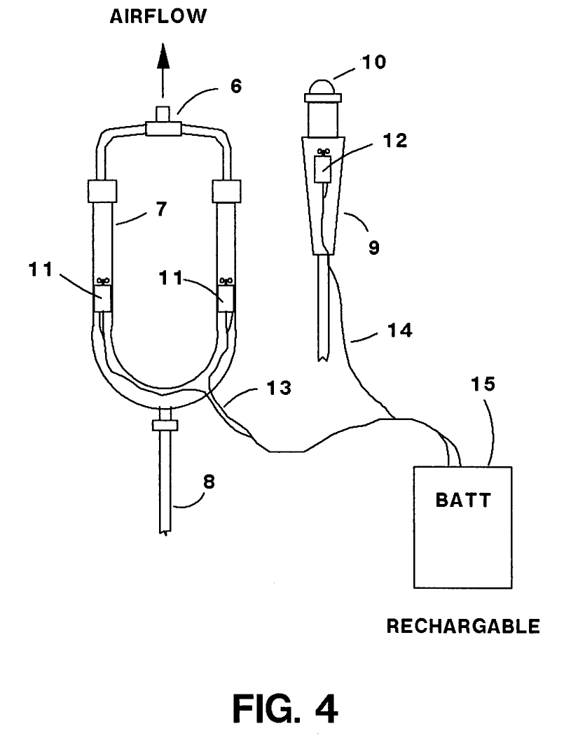

FIG. 4 shows a cut-away detail of the embodiment of FIG. 3. The central portable unit 7 can include one or more internal fans 11. The pickup unit 9 can optionally also contain a fan 12. A preferred fan is a miniature DC fan of model number DB04048B12U made by AVC Company of Taiwan. Any similar fan can be used. The fans can be connected to an electrical source such as a battery 15 by internal or external wires 13, 14 or can be run from conventional power. The battery 15 can optionally be rechargeable.

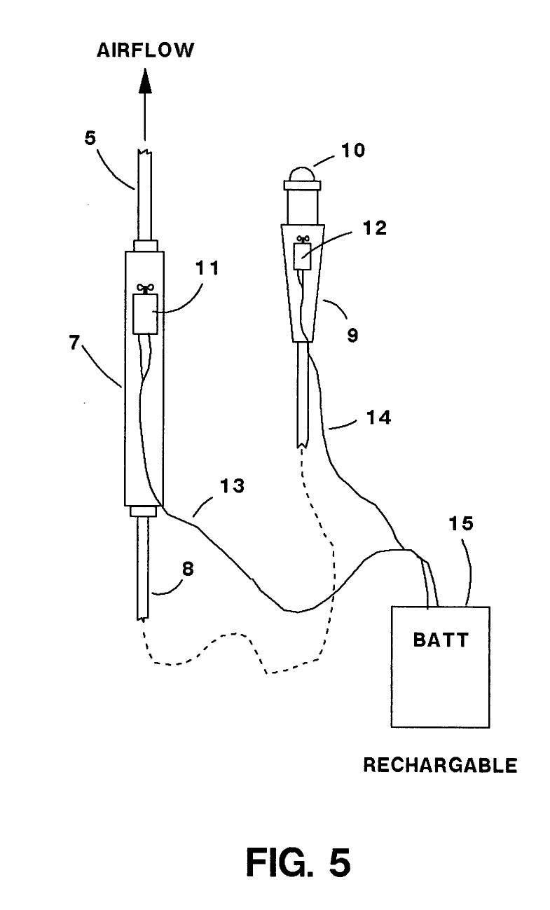

FIG. 5 shows a cut away view of an alternative embodiment. In this embodiment, the air moving chamber 7 includes a single unit with a single fan 11. The hosing is similar in this embodiment to that of the embodiment shown in FIGS. 3-4 except that no T-junction is needed at the air moving chamber. Again, an optionally rechargeable batter 15 can be used. In addition to using a battery, the unit can be operated from line power such as conventional 115 volts.

In the embodiments of FIGS. 3-5, the battery 15 and the air moving unit 7 can be combined into a pack 19 that can be conveniently worn on the waist or back (shown in FIG. 1). Any other type of pack or of carrying the unit is within the scope of the present invention including straps on the air moving unit 7. In yet another embodiment, the battery can be reduced in size and located inside the air moving unit.

In some cases, the search area may be too large or full of materials or there may not be time to search it. In this case, larger fans can be used in the area to direct scents to the portable unit or through a larger hose directly to the animal's nose.

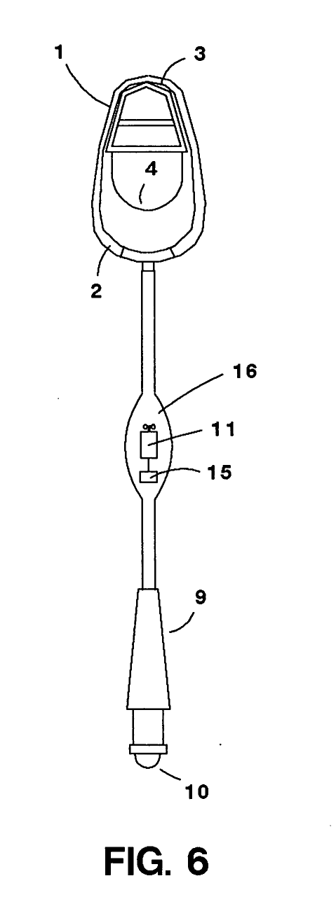

It should be noted in the embodiments of the present invention that the air moving unit can optionally be incorporated directly into an enlarged section of the hose if desired. FIG. 6 shows an embodiment of the invention containing this feature. A widened section of the tubing 7 can contain a miniature fan 11 or a fan 11 and battery 15.

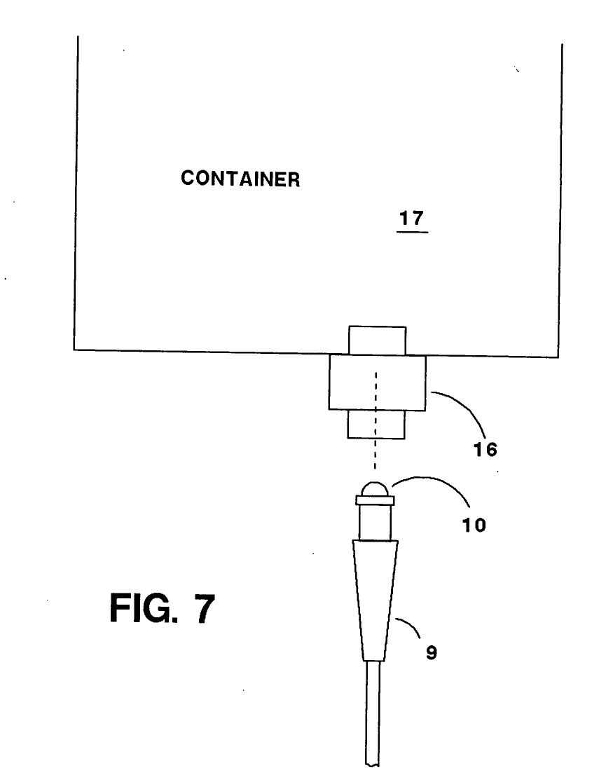

FIG. 7 shows an optional auxiliary external fan unit 16 that has a larger fan or fans that is capable of moving a larger volume of air. This embodiment of the invention is useful for sweeping a large area that is possibly inaccessible such as a cargo container. The larger fan can pull the air out of the container 17 and then directly into the rest of the system as shown in FIG. 7. The external fan unit 16 can be made small enough to carry in the bag 19 (FIG. 1) with the rest of the equipment and plug directly into a fitting in a container or truck. The external fan unit 16 can also be used as a stand-alone unit if desired. The preferred capacity of the external unit is around 400 CFM; however, any capacity is within the scope of the present invention.

It should be noted that the present invention finds many applications beyond the simple searching for contraband. In particular, the invention can be used to sweep for buried land mines, munitions and the like. An operator can use an extension to move the probe 10 out ahead of the dog by a reasonably safe distance. In this way, an operator and dog team can advance slowly in an area with buried mines sweeping ahead of them without risking the safety of the operator or animal. In particular, it can also be used to sample re-circulated air on an aircraft such as after boarding using the craft's cabin air system. In this way, an explosives check could be made on the craft before it leaves the gate.

Several descriptions and illustrations have been provided to better aid in understanding the present invention. A person with skill in the art will realize that numerous changes and variations are possible without departing from the spirit of the invention. Each of these changes and variations is within the scope of the present invention.

I claim:

1. A portable scent detection system for moving air from a search area to an animal's nose comprising:
a hand-held pickup for collecting air;
an air moving unit containing at least one fan coupled to said hand-held pickup;
a scent presentation device worn on said animal's head with at least one presentation port near the animal's nose;
a feed hose coupled between the air moving unit and the application unit;
wherein air enters said pickup, flows through said return hose, through said air moving unit, through said feed hose, into said scent application device and into said animal's nose.

2. The portable scent detection system of claim 1 further comprising a fan in said pickup device.

3. The portable scent detection system of claim 1 further comprising a removable external fan for sweeping a larger area.

4. The portable scent detection system of claim 1 further comprising at least two fans in said air moving unit.

5. The portable scent detection system of claim 1 wherein said air moving unit comprises two parallel sections, each of said sections containing a fan.

6. The portable scent detection system of claim 1 wherein said pickup contains a filter.

7. The portable scent detection system of claim 1 further comprising a battery for running said fan.

8. The portable scent detection system of claim 7 wherein said battery is in said air moving unit.

9. The portable scent detection system of claim 1 wherein said air moving unit is a widened section of hose between said feed hose and said return hose.

10. A canine scent detection system comprising in combination a pickup adapted to be held by a human operator for searching out scents connected via a first hose to an air moving unit containing at least one fan further coupled via a second hose to a scent presentation device in proximity to a canine's nose.

11. The canine scent detection system of claim 10 wherein said scent presentation device is worn on said canine's head.

12. The canine scent detection system of claim 10 further comprising a battery powering said fan.

13. The canine scent detection system of claim 10 further comprising two fans in said air moving unit.

14. The canine scent detection system of claim 10 wherein said air moving unit is a widened section of said first and second hoses.

15. The canine scent detection system of claim 10 wherein said widened section of said first and second hoses also contains a battery.

16. The canine scent detection system of claim 10 further comprising running the fan from conventional line current.

17. A method of directing scent from a target area to a canine's nose comprising the steps of:
providing a head-mounted scent presentation unit worn by said canine;
providing a scent pickup unit;
providing a fan gas-coupled between said pickup unit and said scent presentation unit, wherein said fan moves air from said scent pickup unit to said scent presentation unit.

18. The method of claim 17 wherein said fan is battery powered.

19. The method of claim 17 further including the step of using said pickup unit to probe a target area.

20. The method of claim 17 further including a fan in said scent pickup.

21. The method of claim 17 further including providing an external fan that can direct air into said scent pickup unit from an enclosed space.

22. The method of claim 17 wherein said fan is battery powered by a rechargeable battery.
